(19) United States
(12) Patent Application Publication
(10) Pub. No.: US 2025/0049740 A1
(43) Pub. Date: Feb. 13, 2025

(54) COMPOUND AND USE THEREOF IN PREPARATION OF MEDICAMENT FOR TREATING ALZHEIMER'S DISEASE

(71) Applicant: TAO SUN, Xiamen (CN)

(72) Inventor: TAO SUN, Xiamen (CN)

(21) Appl. No.: 18/923,008

(22) Filed: Oct. 22, 2024

(30) Foreign Application Priority Data
Sep. 26, 2024 (CN) 2024113507770

(51) Int. Cl. A61K 31/167; A01K 67/027; A61P 1/00; A61P 25/28; A61P 29/00

(52) U.S. Cl. CPC A61K 31/167; A01K 67/027; A61P 1/00; A61P 25/28; A61P 29/00; A01K 2207/20; A01K 2227/105; A01K 2267/0318; A01K 2267/0368

(57) ABSTRACT

The present disclosure relates to a compound and the use thereof in the preparation of a medicament for treating Alzheimer's disease. The present disclosure provides a method for constructing an animal model of diseases with N-oleoyl-dopamine (OLDA) and the use of the model in drug screening and animal experimental research, wherein the animal model includes at least one of an animal model of neurodegenerative diseases, an animal model of intestinal permeability diseases, and an animal model of inflammatory diseases. The present disclosure is based on the discovery that OLDA has a new use for a neurodegenerative disease, an intestinal permeability disease, and an inflammatory disease, and based on OLDA, compounds that have a therapeutic effect on the above diseases are identified.

### CROSS-REFERENCE TO RELATED APPLICATION

This application claims the benefit of priority to Chinese Patent Application No. 202411350777.0, filed Sep. 26, 2024, the entire disclosure of which is incorporated herein by reference.

### TECHNICAL FIELD

The present disclosure relates to the technical field of medicine, in particular to a compound and the use thereof in the preparation of a medicament for treating Alzheimer's disease.

### BACKGROUND

Alzheimer's Disease (AD) is a chronic neurodegenerative disease, which is the most common and typical form of dementia. The clinical diagnosis of AD is usually based on the neuropathological features of brain tissues, including senile plaques formed by abnormal deposition of beta-Amyloid protein (Aβ) and neurofibrillary tangles formed by abnormal phosphorylation of Tau protein. The main symptoms of AD include memory loss, cognitive decline, language barriers, changes in mood and behaviors, and decline in daily life skills. With the acceleration of population aging, epidemiological predictions also suggest that drugs that can effectively prevent the onset, delay the progress, or improve the symptoms of AD are an urgent need at present.

AD drugs of small molecules that have been approved by the U.S. Food and Drug Administration include donepezil, rivastigmine, galanthamine, meridinol, etc. These drugs are mainly aimed at improving cognitive symptoms, but neither results in the pathological changes in the brain of AD patients nor slows down or stops the progress of the disease. It is necessary to develop new drugs from the perspective of improving the pathological changes of AD.

### SUMMARY

The present disclosure provides a compound and the use thereof in the preparation of a medicament for treating Alzheimer's disease.

In the first aspect, the present disclosure provides the use of N-oleoyl-dopamine (OLDA) in the construction of an animal model of diseases. The animal model of diseases includes, but is not limited to, an animal model of neurodegenerative diseases, an animal model of intestinal permeability diseases, and an animal model of inflammatory diseases, etc.

In some embodiments of the present disclosure, the animal model relates to a non-human mammal, including a rodent (such as a mouse), a primate, etc.

In some embodiments of the present disclosure, the neurodegenerative diseases include, but are not limited to, diseases such as Alzheimer's disease, Parkinson's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, diseases such as neuroinflammation, inflammatory arthropathy, allergy, autism, anxiety, depression, and chronic fatigue syndrome, wherein the neuroinflammation may be specifically brain neuroinflammation.

In some embodiments of the present disclosure, a mode of administration includes, but is not limited to, oral administration, injection administration, etc.

In some embodiments of the present disclosure, the animal model of diseases is used for animal experimental research, drug screening, etc.

In the second aspect, the present disclosure provides a method for constructing an animal model of diseases, comprising administering an effective dose of N-oleoyl-dopamine to an animal, wherein the animal model of diseases includes an animal model of neurodegenerative diseases, an animal model of intestinal permeability diseases, and an animal model of inflammatory diseases.

In some embodiments of the present disclosure, the animal model relates to a non-human mammal, including a rodent (such as a mouse), a primate, etc.

In some embodiments of the present disclosure, the neurodegenerative diseases include, but are not limited to, diseases such as Alzheimer's disease, Parkinson's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, diseases such as neuroinflammation, inflammatory arthropathy, allergy, autism, anxiety, depression, and chronic fatigue syndrome, wherein the neuroinflammation may be specifically brain neuroinflammation.

In some embodiments of the present disclosure, a mode of administration includes, but is not limited to, oral administration, injection administration, etc.

In some embodiments of the present disclosure, the effective dose is 0.5 mg/kg to 1.5 mg/kg

In the third aspect, the present disclosure provides the use of a compound or a pharmaceutically acceptable salt, hydrate, solvate, racemate, enantiomer, diastereomer, or polymorph thereof in the prevention or treatment of a disease in an animal or in the preparation of a medicament for preventing or treating a disease in an animal, wherein the compound has the following chemical structure of formula I:

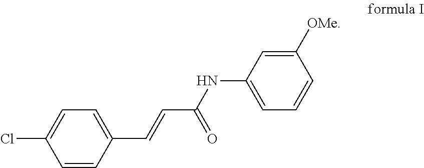 [SMILES: COc1cccc(NC(=O)/C=C/c2ccc(Cl)cc2)c1]

The diseases include, but are not limited to, neurodegenerative diseases, intestinal permeability diseases, and inflammatory diseases, etc.

In some embodiments of the present disclosure, the neurodegenerative diseases include, but are not limited to, diseases such as Alzheimer's disease, Parkinson's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, diseases such as neuroinflammation, inflammatory arthropathy, allergy, autism, anxiety, depression, and chronic fatigue syndrome, wherein the neuroinflammation may be specifically brain neuroinflammation.

In some embodiments of the present disclosure, the occurrence, development, metastasis, or recurrence, etc. of the above diseases may be mediated by N-oleoyl-dopamine.

In some embodiments of the present disclosure, the animal includes a mammal, including a primate, including a human being.

In some embodiments of the present disclosure, the mode of administration includes, but is not limited to, oral administration, injection administration, etc.

In some embodiments of the present disclosure, the compound or the pharmaceutically acceptable salt, hydrate, solvate, racemate, enantiomer, diastereomer, or polymorph thereof is administered as a pharmaceutical composition, wherein the pharmaceutical composition further comprises a pharmaceutically acceptable excipient.

The pharmaceutically acceptable excipient includes, but is not limited to, a carrier, a diluent, a stabilizer, a colorant, a solvent, a chelating agent, a dispersant, a preservative, an antifreeze agent, a thickener, a pH regulator, a protective agent, a tension regulator, etc.

The composition includes, but is not limited to, any one of a liquid preparation, a solid preparation, a sustained-release preparation, and a controlled-release preparation.

In the fourth aspect, the present disclosure provides a method for preventing or treating a disease in an animal, the method comprising administering an effective dose of a compound or a pharmaceutically acceptable salt, hydrate, solvate, racemate, enantiomer, diastereomer, or polymorph thereof to the animal, wherein the compound has the following chemical structure of formula I:

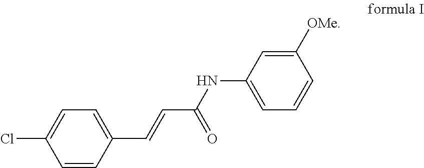 [SMILES: COc1cccc(NC(=O)/C=C/c2ccc(Cl)cc2)c1]

The diseases include, but are not limited to, neurodegenerative diseases, intestinal permeability diseases, and inflammatory diseases, etc.

In some embodiments of the present disclosure, the neurodegenerative diseases include, but are not limited to, diseases such as Alzheimer's disease, Parkinson's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, diseases such as neuroinflammation, inflammatory arthropathy, allergy, autism, anxiety, depression, and chronic fatigue syndrome, wherein the neuroinflammation may be specifically brain neuroinflammation.

In some embodiments of the present disclosure, the animal includes a mammal, including a primate, including a human being.

In some embodiments of the present disclosure, the mode of administration includes, but is not limited to, oral administration, injection administration, etc.

In some embodiments of the present disclosure, the compound or the pharmaceutically acceptable salt, hydrate, solvate, racemate, enantiomer, diastereomer, or polymorph thereof is administered as a pharmaceutical composition, wherein the pharmaceutical composition further comprises a pharmaceutically acceptable excipient.

The pharmaceutically acceptable excipient includes, but is not limited to, at least one of a carrier, a diluent, a stabilizer, a colorant, a solvent, a chelating agent, a dispersant, a preservative, an antifreeze agent, a thickener, a pH regulator, a protective agent, a tension regulator, etc.

The composition includes, but is not limited to, any one of a liquid preparation, a solid preparation, a sustained-release preparation, and a controlled-release preparation.

In the fifth aspect, the present disclosure provides the use of a composition in the prevention or treatment of a disease in an animal or in the preparation of a medicament for preventing or treating a disease in an animal, wherein the composition comprising:

A compound or a pharmaceutically acceptable salt, hydrate, solvate, racemate, enantiomer, diastereomer, or polymorph thereof, and a pharmaceutically acceptable excipient;

The compound has the following chemical structure of formula I:

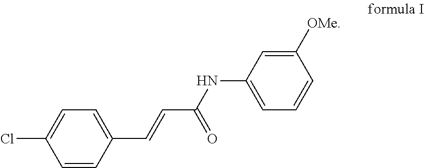 [SMILES: COc1cccc(NC(=O)/C=C/c2ccc(Cl)cc2)c1]

The diseases include, but are not limited to, neurodegenerative diseases, intestinal permeability diseases, and inflammatory diseases, etc.

In some embodiments of the present disclosure, the pharmaceutically acceptable excipient comprised in the composition includes, but is not limited to, a carrier, a diluent, a stabilizer, a colorant, a solvent, a chelating agent, a dispersant, a preservative, an antifreeze agent, a thickener, a pH regulator, a protective agent, a tension regulator, etc.

In some embodiments of the present disclosure, the composition includes, but is not limited to, pharmaceutically acceptable dosage forms such as a liquid preparation, a solid preparation, a sustained-release preparation, and a controlled-release preparation.

In some embodiments of the present disclosure, the neurodegenerative diseases include, but are not limited to, diseases such as Alzheimer's disease, Parkinson's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, diseases such as neuroinflammation, inflammatory arthropathy, allergy, autism, anxiety, depression, and chronic fatigue syndrome, wherein the neuroinflammation may be specifically brain neuroinflammation.

In some embodiments of the present disclosure, the animal includes a mammal, including a primate, including a human being.

In some embodiments of the present disclosure, the mode of administration includes, but is not limited to, oral administration, injection administration, etc.

The present disclosure is based on the discovery that OLDA has a new use for a neurodegenerative disease, an intestinal permeability disease, and an inflammatory disease, and based on OLDA, compounds that play an active role in the above diseases are screened out.

Additional aspects and advantages of the present disclosure will be set forth in part in the description below and in part will be obvious from the description below, or may be learned by the practice of the present disclosure.

### BRIEF DESCRIPTION OF DRAWINGS

FIG. 1: an analysis diagram of plasma metabolites. Among the diagram, panel A and panel B of FIG. 1 are respectively a principal component analysis diagram of C57-Ctrl and C57-AD-FMT mice and the statistical analysis results of differential metabolites, and panel C of FIG. 1 is a heat map of the analysis of plasma metabolites in mice in different groups.

FIG. 2: a schematic diagram of grouping of experimental mice in Example 3.

FIG. 3: a statistical diagram of a mouse behavioral experiment. Among the diagram, panel A and panel B of FIG. 3 are the spontaneous alternation rate (% of Alternation) and the total number of arm entries (total entries) of mice; panel C of FIG. 3 is the escape latency for the mice to find a platform if the platform is present; and panel D of FIG. 3 is the number of times the mice cross the original platform location when the platform is absent (platform crossings).

FIG. 4: immunohistochemical images of Aβ42 (panel A of FIG. 4) and p-Tau181 (panel C of FIG. 4) in the hippocampus of the mouse brain, both with the scale of 250 μm. Panel B and panel D of FIG. 4 are respectively the statistical analysis results of panel A and panel C of FIG. 4.

FIG. 5: immunohistochemical images of IBA-1 (panel A of FIG. 5) and GFAP (panel C of FIG. 5) in the hippocampus of mice, both with the scale of 250 μm. Panel B and panel D of FIG. 5 are respectively the statistical analysis results of panel A and panel C of FIG. 5.

FIG. 6: HE staining images of mouse colon tissues (panel A of FIG. 6, scale: 250 μm), and panel B of FIG. 6 is the comparison results of histological scores in panel A of FIG. 6.

FIG. 7: a statistical diagram showing a compound of formula I for treating cognitive behavioral abnormalities in AD mice. Among the diagram, panel A and panel B of FIG. 7 are the spontaneous alternation rate (% of Alternation) and the total number of arm entries (total entries) of mice; panel C of FIG. 7 is the escape latency for the mice to find a platform if the platform is present; and panel D of FIG. 7 is the number of times the mice cross the original platform location when the platform is absent (platform crossings).

Note: Each point in the statistical diagram represents an experimental mouse. The statistical method is a t test, with means±SEM, *p<0.05, **p<0.01, ***p<0.001, and ns means no significant difference (no significance).

### DETAILED DESCRIPTION

Exemplary embodiments of the present invention are set forth below. It should be recognized that the description is not intended as a limitation on the scope of the present invention but is provided as a description of exemplary embodiments.

In the description of the present disclosure, the term several means one or more, the term multiple means two or more, the terms more than, less than, exceeding, etc. are understood as excluding the stated number, and the terms not less than, not more than, within, etc. are understood as including the stated number. If the terms first and second are described, they are only used for the purpose of distinguishing technical features and cannot be understood as indicating or implying relative importance, or implicitly indicating the number of indicated technical features, or implicitly indicating the sequence of indicated technical features.

Unless otherwise defined, all the technical and scientific terms used in the present disclosure have the same meanings as commonly understood by those skilled in the technical field to which the present disclosure belongs. The terms used herein are only for the purpose of describing examples of the present disclosure and are not intended to limit the present disclosure.

In the description of the present disclosure, descriptions with reference to the terms “one example”, “some examples”, “exemplary examples”, “instances”, “specific instances”, or “some instances”, etc. mean that specific features, structures, materials, or characteristics described in connection with the example or instance are included in at least one example or instance of the present disclosure. In the description, the schematic expression of the above terms does not necessarily refer to the same example or instance. Moreover, the specific features, structures, materials, or characteristics described may be combined in any one or more examples or instances in a suitable manner.

N-oleoyl-dopamine (OLDA) is a fatty acid derivative of dopamine, which is produced by combining dopamine with oleic acid through a direct N-acylation reaction. There are two sources for OLDA: human cells produce OLDA through the hydrolysis of N-acyltransferase or phospholipids; or OLDA is produced by specific intestinal symbiotic bacteria ^{[1]}. Studies have shown that OLDA is endogenous in mammalian brains, and the metabolism thereof may follow a similar pathway to dopamine, especially through an O-methylation process catalyzed by catechol-O-methyltransferase ^{[2]}. However, there is no report about the relationship between OLDA and neurodegenerative diseases, such as Alzheimer's disease and Parkinson's disease, and other related diseases with similar pathologies or conditions.

Therefore, the present disclosure provides the first aspect of the present disclosure according to the relevant experimental results, which relates to the use of N-oleoyl-dopamine in the construction of an animal model of diseases and the use of the model in animal experimental research and drug screening, wherein the animal models include, but are not limited to, animal models of a neurodegenerative disease, an intestinal permeability disease, an inflammatory disease, etc.

The term “animal model of diseases” refers to a diseased animal model that reproduces the onset and development of a human disease in the experimental animal model, either partially or fully, through methods such as induction, surgery, or genetic editing. In addition, the use of N-oleoyl-dopamine in the construction of an animal model in the present disclosure refers to using compounds including N-oleoyl-dopamine as one of the inducing means to act on experimental animals, causing some damage to their tissues, organs or whole body to some extent and changes in functions, metabolism, morphology, etc. It can be understood that other physical, chemical, and biological methods, etc., can also be used alone or in combination as inducing means.

The experimental animals for animal models are usually derived from mammals. Since the animal model is used for reproducing the relevant process of a human disease, animals for the animal model include non-human mammals, specifically including experimental animals with clear genetic pedigree and clear legal sources, which are well known to and applied by those skilled in the art, such as murine, dogs, monkeys, rabbits, and pigs. Considering the genetic background, growth period, operability, etc., mice, rats, guinea pigs, beagle dogs, China white rabbits, New Zealand white rabbits, rhesus monkeys, cynomolgus monkeys, etc. are more commonly used.

The term “neurodegenerative diseases” refers to a disease caused by gradual degeneration of spinal cord and brain neurons, including acute neurodegenerative diseases and chronic neurodegenerative diseases. The former includes cerebral ischemia (CI), brain injury (BI), epilepsy, etc., and the latter includes Alzheimer's disease (AD), Parkinson's disease (PD), multiple sclerosis (MS), amyotrophic lateral sclerosis (ALS), spinocerebellar ataxia (SCA), dementia of Pick's disease, etc.

The term “intestinal permeability” is a measurement feature of intestinal barrier. Intestinal permeability refers to a process of molecules passing through the intestinal epithelial cell layer in a carrier-independent diffusion manner and is a barrier function closely related to intestinal symbiotic flora and mucosal immune system. Changes in intestinal permeability allow antigens in the intestinal cavity to penetrate intestinal mucosa disproportionately, causing immune dysregulation and resulting in gastrointestinal diseases or systemic immune diseases, such as inflammatory bowel disease, irritable bowel syndrome, celiac disease, obesity, non-alcoholic fatty liver disease, type 2 diabetes, cardiovascular diseases, and cancer. Changes in intestinal permeability caused by intestinal mucosa damage are the pathological characteristics of inflammatory bowel diseases, including Crohn's disease and ulcerative colitis ^{[3,4]}. Increased intestinal permeability in patients with irritable bowel syndrome is related to immune activation, inflammatory factors, etc., which in turn increases intestinal permeability ^{[5]}. Obesity and related metabolic diseases, such as pathophysiological changes, e.g., non-alcoholic fatty liver, type 2 diabetes, and cardiovascular diseases, are all related to changes in intestinal permeability ^{[6]}. Other cancers, such as colon cancer, have also been proved to be closely related to changes in intestinal permeability. Therefore, by increasing or decreasing intestinal permeability, the occurrence and development of the above-mentioned intestinal permeability diseases can be induced or alleviated.

The term “inflammation” refers to a vascular-centered defensive response produced in tissues with blood vessels to the stimulation of inflammatory factors, including biological factors, physical and chemical factors, abnormal immune responses, necrotic tissues, foreign bodies, etc. Accordingly, an inflammatory disease refers to a disease that comprises inflammations in pathogenic factor and disease progressions and includes, but is not limited to, neuroinflammation, inflammatory arthropathy, allergy (such as food allergy), autism, anxiety, depression, chronic fatigue syndrome, etc. In addition, asthma, chronic sinusitis (CRS), allergic rhinitis (AR), eosinophilic esophagitis (EoE), etc. are also included. In some embodiments, animal models of inflammatory diseases include at least one of an animal model of neuroinflammation, an animal model of inflammatory arthropathy, an allergic model, an autism model, an anxiety model, a depression model, and a chronic fatigue syndrome model, for example, it may be an animal model of brain neuroinflammation.

Accordingly, the present disclosure provides a method for constructing the above-mentioned animal models, comprising administering an effective dose of N-oleoyl-dopamine to an animal, thereby chemically intervening the animal to complete modeling. It can be understood that the above-mentioned chemical intervention means may also further include other chemical reagents or be combined with physical intervention and biological intervention methods and means to obtain better modeling effects.

In some embodiments, the mode of administration of N-oleoyl-dopamine includes, but is not limited to, one or more of oral administration, injection administration, etc. In some embodiments, the number of times of administration may be one or more; the administration interval may be once every 6 hours to 48 hours; and the effective dose of N-oleoyl-dopamine is 0.5 mg/kg to 1.5 mg/kg.

Therefore, in the third aspect of the disclosure, a compound with therapeutic effects on a related disease or a pharmaceutically acceptable salt, hydrate, solvate, racemate, enantiomer, diastereomer, or polymorph thereof is specifically constructed, and the compound has the following structure of formula:

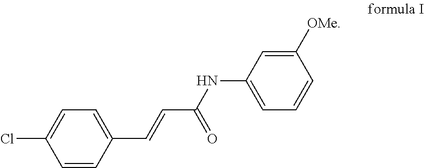 [SMILES: COc1cccc(NC(=O)/C=C/c2ccc(Cl)cc2)c1]

The above compound involved in the examples of the present disclosure can be prepared by the following method, which can be carried out with reference to a synthesis method well known to professionals ^{[7-9]}, and the synthesis method is readily conceivable to professionals in the related art.

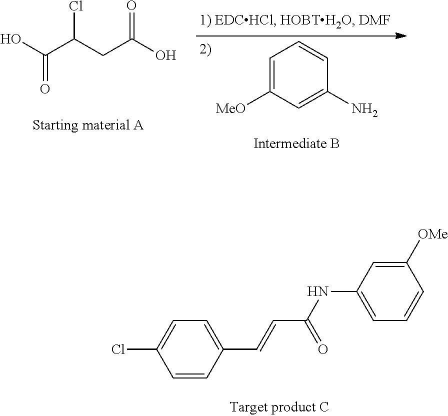 [SMILES: O=C(O)CC(Cl)C(=O)O ; COc1cccc(N)c1 ; COc1cccc(NC(=O)/C=C/c2ccc(Cl)cc2)c1]

Starting material A is taken and dissolved in a solvent to form a first solution, to which EDC·HCl and HOBT·H₂O are added and stirred at room temperature for a period of time, and intermediate B is then added and stirred at room temperature for a period of time to generate the desired target product C.

The present disclosure further relates to a composition comprising the above-mentioned compound or a pharmaceutically acceptable salt, hydrate, solvate, racemate, enantiomer, diastereomer, or polymorph thereof, and a pharmaceutically acceptable excipient, which is for example a mixture obtained by uniformly mixing the two or a mixture obtained by means of other formation. In some embodiments thereof, the pharmaceutically acceptable excipient comprised in the composition includes, but is not limited to, at least one of a carrier, a diluent, a stabilizer, a colorant, a solvent, a chelating agent, a dispersant, a preservative, an antifreeze agent, a thickener, a pH regulator, a protective agent, a tension regulator, etc.

In some embodiments, the dosage form of the composition includes, but is not limited to, a liquid preparation, a solid preparation, etc. The specific dosage form of the liquid preparation may be an injection, a solution, etc. The specific dosage form of the solid preparation may be capsules, tablets, etc. In some embodiments, the dosage form of the composition is any one of a sustained-release preparation and a controlled-release preparation, for example, it may be sustained-release and controlled-release matrix tablets, sustained-release and controlled-release capsules, etc. In some embodiments, the use of the composition includes, but is not limited to, medicines, health care products, etc.

The above compounds can be used as individual compounds and/or mixed with acceptable excipients as pharmaceutical preparations for treating diseases such as neurodegenerative diseases, intestinal permeability diseases, and inflammatory diseases. Those skilled in the art would easily understand the dosage and route of administration of the compound to human beings and/or mammals in need of such treatment. The route of administration may include, but is not limited to, oral administration, intravenous injection, etc. According to the route of administration, the compound preparation is prepared according to instructions of acceptable pharmaceutical operation procedures ^{[10,11]}.

In some embodiments, the compound or a pharmaceutically acceptable salt, hydrate, solvate, racemate, enantiomer, diastereomer, or polymorph thereof are administered in various dosage forms (including immediate-release, sustained-release, or controlled-release dosage forms), such as tablets, capsules, and injections. The compound may be administered alone, or the compound may be generally prepared into corresponding preparation dosage forms for administration according to the selected route of administration and standard pharmaceutical operation procedures.

The above-mentioned dosage regimen will vary depending on known factors, e.g., the pharmacokinetic characteristics of a specific agent, the mode and route of administration thereof, the species, age, sex, health, medical status, and weight of the subject, the nature and degree of symptoms, the category of a concurrent treatment, the frequency of administration, the route of administration, the kidney and liver functions of the patient, and the required therapeutic effect. Physicians or veterinarians can prescribe an effective amount of medication based on the need to prevent, treat, or inhibit the progression of the disease condition.

In these pharmaceutical compositions, the active ingredient will be generally present in an amount of about 0.01% to 95% by weight based on the total weight of the composition.

In some embodiments, compound preparations are generally prepared by selecting suitable excipients depending on the dosage form according to conventional pharmaceutical operation procedures and mixing them evenly. For example, for tablets or capsules, the active pharmaceutical ingredient can be combined with one or more pharmaceutically acceptable excipients such as but not limited to starch, sucrose, methylcellulose, and magnesium stearate. For liquid preparations for oral administration, the excipient component thereof can be combined with one or more of any pharmaceutically acceptable excipients or carriers such as but not limited to one or more of ethylene glycol, water, etc. If necessary, suitable excipients such as a binder, a flavoring agent, a lubricant, a disintegrant, and a pigment may also be added. Suitable binders include, but are not limited to, starch, gelatin, carboxymethyl cellulose, polyethylene glycol, etc.; flavoring agents are for example, but not limited to, glucose, corn sweetener, etc.; lubricants include, but are not limited to, sodium stearate, magnesium stearate, etc.; disintegrants include, but are not limited to, starch methyl cellulose, etc.; and pigments include, but are not limited to, carmine, carotene, etc.

In some embodiments, tablets or capsules, or both, can be manufactured in the form of sustained-release products, thus providing continuous release of drugs over a period of several hours or more. Tablets may be sugar-coated or film-coated, so as to mask unpleasant taste and isolate the tablets from the air, or may be enteric-coated for selective disintegration and release in the gastrointestinal tract. In some embodiments, pigments and flavoring agents may be added to the liquid preparation for oral administration to improve the compliance of medication.

Suitable pharmaceutical excipients are described in the book ^{[10]}, and this reference is a standard reference text in the art and is well known to professional technicians in pharmacy or related fields.

An example of the present disclosure further relates to the use of the above-mentioned compound of formula I or a pharmaceutically acceptable salt, hydrate, solvate, racemate, enantiomer, diastereomer, or polymorph thereof, or the above-mentioned composition in the prevention or treatment of a disease in an animal or the preparation of a medicament for preventing or treating a disease in an animal, comprising administering an effective dose of the above-mentioned compound of formula I or a pharmaceutically acceptable salt, hydrate, solvate, racemate, enantiomer, diastereomer, or polymorph thereof to the animal.

The diseases include, but are not limited to, neurodegenerative diseases, intestinal permeability diseases, and inflammatory diseases, etc.

The neurodegenerative diseases include, but are not limited to, diseases such as Alzheimer's disease, Parkinson's disease, Huntington's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, neuroinflammation, inflammatory arthropathy, allergy, autism, anxiety, depression, chronic fatigue syndrome, etc.

In some embodiments, for the above diseases, the occurrence, development, metastasis or recurrence, etc. of the diseases may be mediated by N-oleoyl-dopamine. In some other embodiments, the above-mentioned diseases may also be those that occur, develop, metastasize, or recur under the action of one or more intrinsic or extrinsic factors such as genetics or environment, without intervention or mediation by N-oleoyl-dopamine.

In some embodiments, the animal includes a mammal, such as a primate, or it may also be a human being, including but not limited to other human individuals at different ages, such as children and adults.

In some embodiments, the mode of administration includes, but is not limited to, oral administration, injection administration, etc.

The present disclosure further relates to a method for preventing or treating a neurodegenerative disease, an intestinal permeability disease, or an inflammatory disease, comprising administering an effective dose of the above-mentioned compound of formula I or a pharmaceutically acceptable salt, hydrate, solvate, racemate, enantiomer, diastereomer, or polymorph thereof, or the above-mentioned composition to the animal.

In some embodiments, the neurodegenerative diseases include, but are not limited to, diseases such as Alzheimer's disease, Parkinson's disease, Huntington's disease, multiple sclerosis, amyotrophic lateral sclerosis, spinocerebellar ataxia, and dementia of Pick's disease.

The intestinal permeability diseases include, but are not limited to, diseases such as inflammatory bowel disease, irritable bowel syndrome, celiac disease, obesity, non-alcoholic fatty liver disease, type 2 diabetes, cardiovascular disease, and cancer.

The inflammatory diseases include, but are not limited to, diseases such as neuroinflammation, inflammatory arthropathy, allergy, autism, anxiety, depression, and chronic fatigue syndrome.

In some embodiments, the animal includes a mammal, such as a primate, or it may be specifically a human being, including but not limited to other human individuals at different ages, such as children and adults.

In some embodiments, the mode of administration includes, but is not limited to, oral administration, injection administration, etc.

### ABBREVIATIONS

| Abbreviation | Explanation of abbreviation | Abbreviation | Explanation of abbreviation |
|---|---|---|---|
| AD | Alzheimer's disease | FDA | U.S. Food and Drug |
| | | | Administration |
| Aβ | Beta-Amyloid protein | OLDA | N-oleoyl-dopamine |
| IBA-1 | Ionized calcium binding | HE staining | Hematoxylin-eosin staining |
| | adaptor 1 | | |
| GFAP | Glial fibrillary acidic | EDTA | Ethylenediamine tetraacetic |
| | protein | | acid |
| IHC | Immunohistochemistry | FMT | Fecal microbiota |
| | | | transplantation |
| mg | Milligram | g | Gram |
| EDC•HCl | 1-Ethyl-(3- | HOBT | 1-Hydroxybenzotriazole |
| | dimethylaminopropyl)carbo | | |
| | diimide hydrochloride | | |
| DMF | N,N-dimethylformamide | LCMS | Liquid chromatography- |
| | | | mass spectrometry |

### REFERENCES

[1] Chang F Y, Siuti P, Laurent S, et al. Gut-inhabiting Clostridia build human GPCR ligands by conjugating neurotransmitters with diet- and human-derived fatty acids[J]. Nat Microbiol, 2021, 6(6): 792-805.
[2] Zajac D, Spolnik G, Roszkowski P, et al. Metabolism of N-acylated-dopamine[J]. PLoS One, 2014, 9(1): e85259.
[3] Hering N A, Fromm M, Schulzke J D. Determinants of colonic barrier function in inflammatory bowel disease and potential therapeutics[J]. J Physiol, 2012, 590(5): 1035-44.
[4] Duckworth C A, Watson A J. Analysis of epithelial cell shedding and gaps in the intestinal epithelium[J]. Methods Mol Biol, 2011, 763: 105-14.
[5] Camilleri M, Lasch K, Zhou W. Irritable bowel syndrome: methods, mechanisms, and pathophysiology. The confluence of increased permeability, inflammation, and pain in irritable bowel syndrome[J]. Am J Physiol Gastrointest Liver Physiol, 2012, 303(7): G775-85.
[6] Donath M Y, Shoelson S E. Type 2 diabetes as an inflammatory disease[J]. Nat Rev Immunol, 2011, 11(2): 98-107.
[7] Kürti L, Czako B, Corey E J, et al. Strategic applications of named reactions in organic synthesis: background and detailed mechanisms[C], 2005.
[8] Smith M B, March J. March's Advanced Organic Chemistry: Reactions, Mechanisms, and Structure[C], 2001.
[9] Vollhardt K P C, Schore N E. Organic Chemistry: Structure and Function[C], 1987.
[10] Remington J P. Remington: the science and practice of pharmacy[M]. 1. Lippincott Williams & Wilkins, 2006.
[11] Goodman L S, Gilman A, Koelle G B. The pharmacological basis of therapeutics[C], 1975.

### Example 1

This example provided a general method for synthesizing formula I. The preparation method thereof was carried out by reference to the following synthesis route, and the specific steps were as follows:

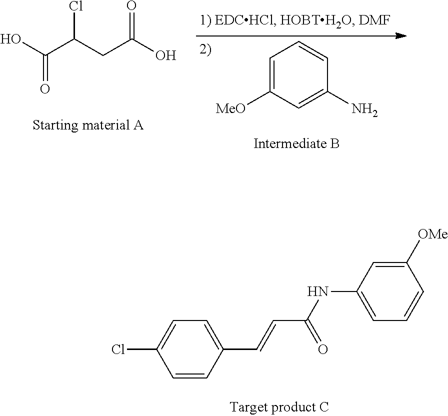 [SMILES: O=C(O)CC(Cl)C(=O)O ; COc1cccc(N)c1 ; COc1cccc(NC(=O)/C=C/c2ccc(Cl)cc2)c1]

(1) 3.0 g of starting material A was added to dimethylformamide (DMF) solvent to form a solution.
(2) 3.45 g of EDC HCl (1-(3-dimethylaminopropyl)-3-ethylcarbodiimide hydrochloride) and 2.76 g of HOBT·H₂O (1-hydroxybenzotriazole hydrate) were added to the solution and stirred at room temperature for 20-30 min.
(3) 2.22 g of intermediate B was added to the mixture in step (2) and continuously stirred for 20-24 hours at room temperature.
(4) After the reaction was completed, the reaction solution was concentrated under reduced pressure.
(4) The concentrated residue was dissolved in ethyl acetate, washed with a 1 N hydrochloric acid solution, then dried with anhydrous sodium sulfate for 24 hours, and concentrated under reduced pressure again.
(5) The concentrate was purified by an alkaline alumina chromatographic column with ethyl acetate as an eluent, and the ethyl acetate eluent was subjected to concentration followed by crystallization to obtain the final product (target product C). LCMS: [M+H]⁺=288.08.

### Example 2: Intestinal flora Transplantation in Mice and Analyses of Plasma Metabolites Thereof

According to the standard operating procedures of intestinal flora transplantation experiment, the experimental mice were divided into the following four groups: normal wild-type mice (C57-AD-FMT) transplanted with intestinal flora of 3×Tg mice (AD mice), wild-type mice as the control group (C57-Ctrl), 3×Tg mice, and wild-type mice (WT) in the same cage of 3×Tg mice. Intestinal flora was collected from the AD mice and transplanted into 4-week-old wild-type mice (C57-AD-FMT). In transplantation week 1, the transplantation was carried out by oral gavage consecutively for the first 6 days and transplanted 4 times a week from week 2. Accordingly, the mice in the control group were given the same volume of a sterile phosphate buffer. Intestinal flora transplantation was carried out for 8 weeks, plasma was collected from the above four groups of experimental mice at the age of 12 weeks, and the metabolites in the plasma samples were analyzed by non-targeted metabolomics technology.

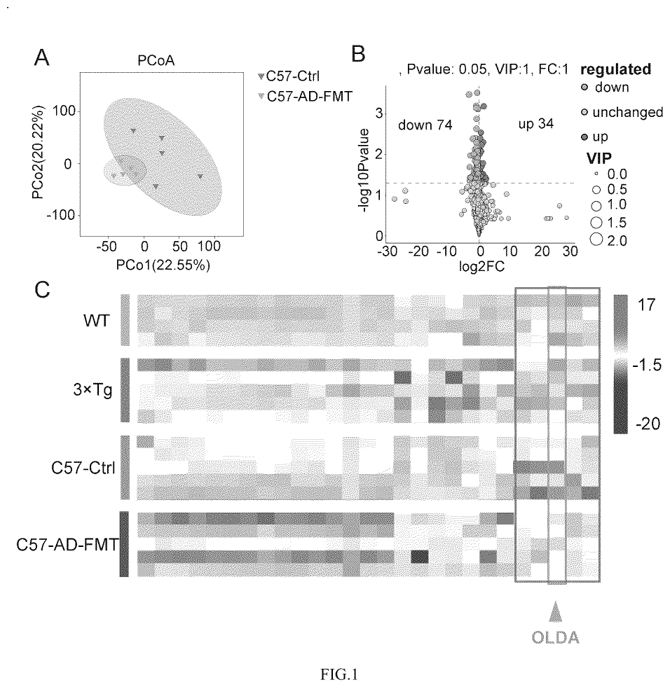

There was a significant difference in metabolism between C57-Ctrl mice and C57-AD-FMT mice (panel A in FIG. 1). The results of differential analysis showed that 74 metabolites were significantly down-regulated, and 34 metabolites were significantly up-regulated, and the differential metabolism was mainly manifested in lipid and fatty amide metabolism disorders (panel B in FIG. 1). Combined analysis of the non-target metabolomic sequencing of the mice in the four groups showed that OLDA was specifically increased not only in the 3×Tg mice but also in the C57-AD-FMT mice (panel C in FIG. 1), indicating that OLDA could play a role during the pathogenesis of AD.

### Example 3: Wild-Type Mice Fed With OLDA and Formula I and Their Behavioral Studies

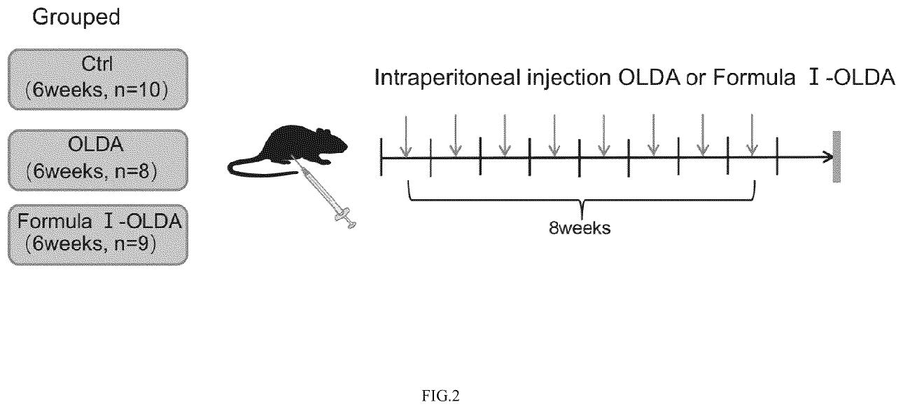

The experimental design and grouping were as shown in FIG. 2, according to the standard operation procedures of mice administration comparison experiment. 6-week-old female wild-type mice C57BL/6J were divided into a control group injected with normal saline (Ctrl), a diseased group (injected with OLDA), and a treatment group (Formula I-OLDA). The administration was carried out by intraperitoneal injection, and the diseased group (OLDA, 0.5 mg/kg to 1.5 mg/kg), the control group (injected with normal saline, the volume of which was equal to that in the diseased group), and the treatment group (0.5 mg/kg to 1.5 mg/kg of OLDA, and 0.5 mg/kg to 1.5 mg/kg of formula I, prepared into a mixed solution for administration) were all administered once a day by injection for 8 consecutive weeks. After administration, the learning and memory ability of the mice in each group was evaluated by Y maze and Morris water maze tests.

Compared with the control group, the spontaneous alternation rate and the total number of arm entries of the mice in the diseased group were both significantly reduced; the time it took to find the platform was significantly prolonged. After the platform was removed, the number of times of crossing the original platform location was also significantly reduced, indicating that OLDA could reduce the learning and memory ability of the mice.

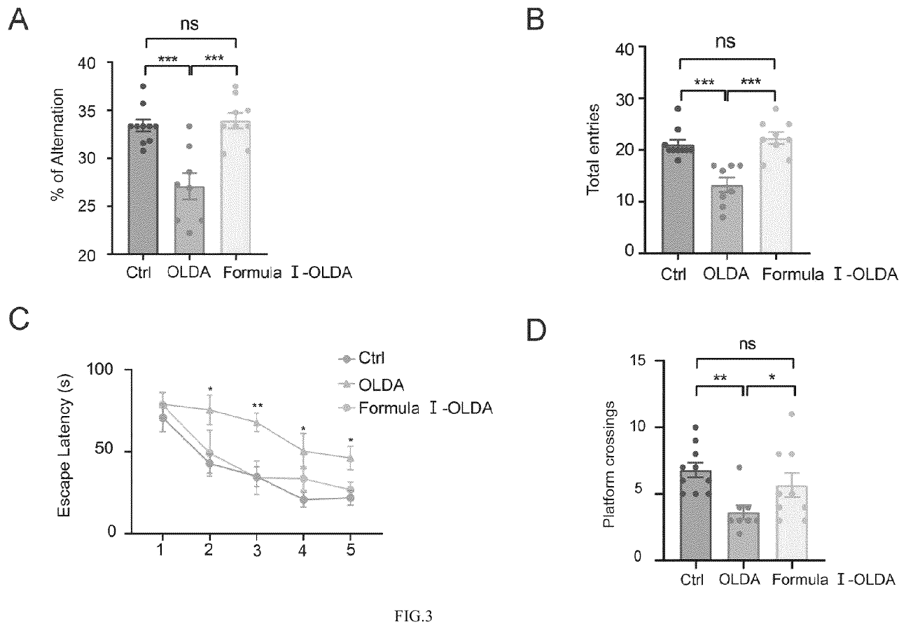

Compared with the diseased group, the spontaneous alternation rate and the total number of arm entries of the mice in the treatment group significantly increased (panel A and panel B in FIG. 3), the time it took to find the platform was significantly reduced (panel C in FIG. 3). After the platform was removed, the number of times of crossing the original platform location significantly increased (panel D in FIG. 3), indicating that formula I could improve the OLDA-induced cognitive dysfunction in the mice.

### Example 4: Dissection and Sampling From Mice in Example 3

According to the standard operating procedure of mouse brain anatomy, the mice in the control group, diseased group and treatment group in Example 3 were sacrificed, and their brain tissues and colon tissues were collected respectively and cryopreserved at −80° C.

### Example 5: Brain Pathological Examination of Wild-Type Mice Fed With OLDA

The brain tissues of the mice in the diseased group and control group in Example 4 were examined by IHC technology for AD-related biochemical markers, and examined by IHC technology for the related markers of brain neuroinflammation.

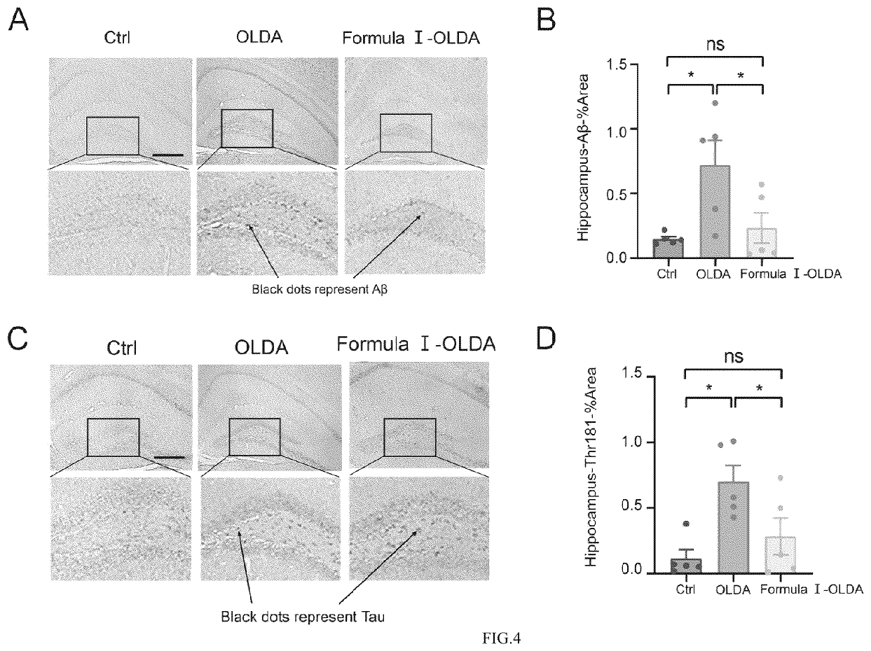

Compared with the control group, the areas of Aβ and p-Tau181 in the hippocampus of the mouse brain in the diseased group significantly increased (FIG. 4), indicating that the continuous administration of OLDA could induce AD symptoms in the mice; and the number of IBA-1 and GFAP positive cells in the mice in the diseased group increased, indicating that the continuous administration of OLDA could induce brain neuroinflammation in mice.

### Example 6: Colon Tissue Examination of Wild-Type Mice Fed With OLDA

The colon tissues in the diseased group and control group in Example 4 were examined by HE staining technology for colonic epithelium histopathology.

Compared with the control group, inflammatory cells in the colon tissue of the mice in the diseased group infiltrated into muscularis mucosa, and the number of crypts decreased, indicating that OLDA could damage the colon tissue of the mice.

### Example 7: Use of Formula I in AD-Related Disease

The brain tissues of the mice in the treatment group in Example 4 were examined by IHC technology for AD-related biochemical markers and compared with the diseased group.

Compared with the diseased group, the areas of Aβ (panel A and panel B in FIG. 4) and p-Tau181 (panel C and panel D in FIG. 4) in the hippocampus of the mouse brain in the treatment group significantly decreased, indicating that formula I could improve the pathological symptoms of OLDA-induced AD.

### Example 8: Use of Formula I for Brain Neuroinflammation

The brain tissues of the mice in the treatment group in Example 4 were examined by IHC technology for the related markers of brain neuroinflammation and compared with the diseased group.

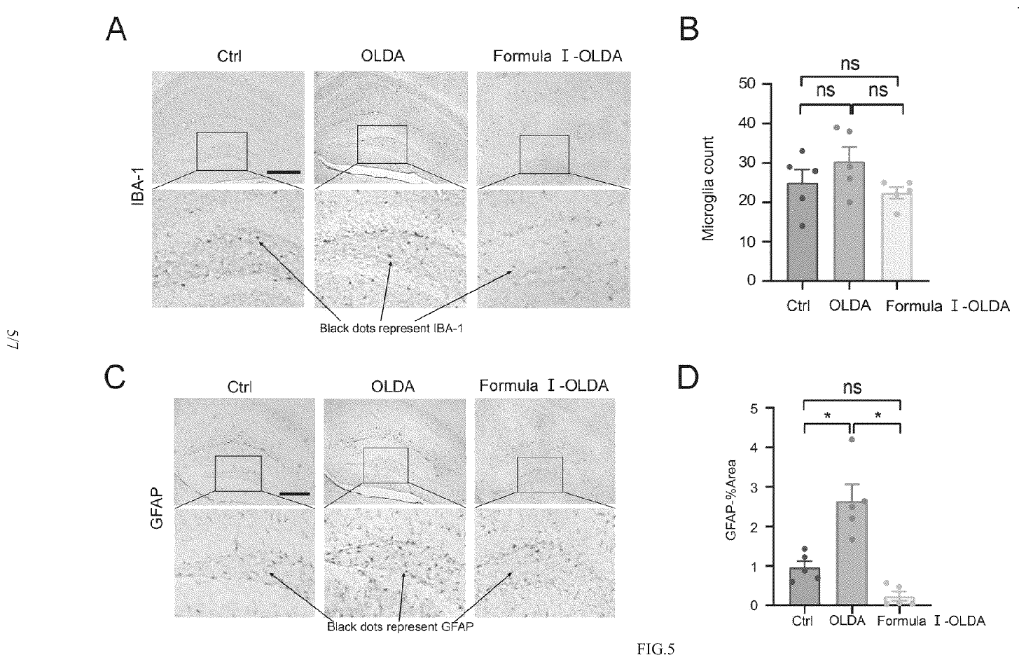

Compared with the diseased group, the number of IBA-1 and GFAP positive cells in the mice in the treatment group decreased (panel A to panel D in FIG. 5), indicating that formula I could improve OLDA-induced brain neuroinflammation.

### Example 9: Use of Formula I for Diseases Related to Intestinal Mucosal Permeability

The colon tissues of the mice in the treatment group in Example 4 were examined by HE staining technology for colonic epithelium histopathology and compared with the diseased group.

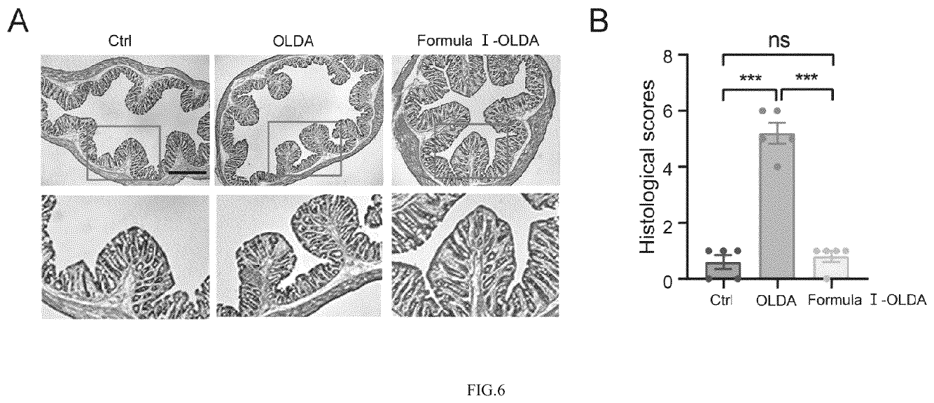

Compared with the diseased group, inflammatory cells in the colon tissue of the mice in the treatment group infiltrated into muscularis mucosae, and the number of crypts increased (panel A and panel B in FIG. 6), indicating that formula I could improve OLDA-induced colonic epithelial injury.

### Example 10

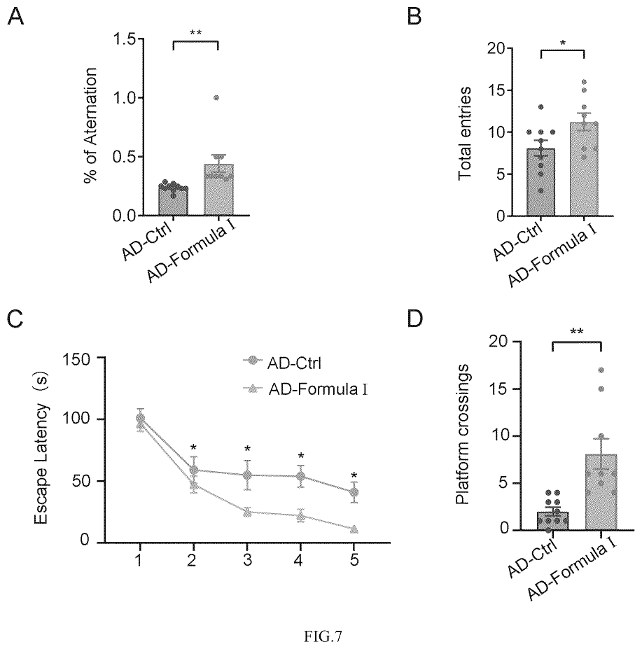

3×Tg mice (AD mice) in Example 2 were directly injected with a composition containing formula I (treatment group). Compared with the control group injected with normal saline, the areas of Aβ and p-Tau181 in the hippocampus of the mice in the treatment group decreased, and the number of IBA-1 and GFAP positive cells decreased, indicating that formula I could treat the pathological symptoms of AD and improve brain neuroinflammation. Inflammatory cells in the colon tissue of the mice in the treatment group infiltrated into muscularis mucosae, and the number of crypts increased, indicating that formula I could improve colonic epithelial injury. In addition, the spontaneous alternation rate and the total number of arm entries of the mice in the treatment group significantly increased (panel A and panel B in FIG. 7), the time it took to find the platform was significantly reduced (panel C in FIG. 7), and after the platform was removed, the number of times of crossing the original platform location was significantly reduced (panel D in FIG. 7), indicating that the compound of formula I could improve the cognitive dysfunction in AD mice.

The present disclosure has been described above in detail in conjunction with examples; however, the present disclosure is not limited to the above examples, and various changes can also be made within the knowledge scope of those of ordinary skills in the pertinent technical field without departing from the gist of the present disclosure. Without conflict, the examples of the present disclosure and the features in the examples can be combined with each other.

1. A method for constructing an animal model of diseases, wherein the method comprises administering an effective dose of N-oleoyl-dopamine to an animal; the animal model of diseases includes an animal model of neurodegenerative diseases, an animal model of intestinal permeability diseases, and an animal model of inflammatory diseases.

2. The method according to claim 1, wherein an animal for the animal model of diseases includes a non-human mammal.

3. The method according to claim 1, wherein an animal for the animal model of diseases includes at least one of a rodent and a primate.

4. The method according to claim 1, wherein an animal for the animal model of diseases includes a mouse.

5. The method according to claim 1, wherein the neurodegenerative diseases include at least one of Alzheimer's disease, Parkinson's disease, multiple sclerosis, amyotrophic lateral sclerosis, spinocerebellar ataxia, and dementia of Pick's disease.

6. The method according to claim 1, wherein the intestinal permeability diseases include at least one of inflammatory bowel disease, irritable bowel syndrome, celiac disease, obesity, non-alcoholic fatty liver disease, type 2 diabetes, cardiovascular disease, and cancer.

7. The method according to claim 1, wherein the inflammatory diseases include at least one of neuroinflammation, inflammatory arthropathy, allergy, autism, anxiety, depression, and chronic fatigue syndrome; optionally, the neuroinflammation includes brain neuroinflammation.

8. The method according to claim 1, wherein a mode of administration includes at least one of oral administration and injection administration.

9. The method according to claim 1, wherein the effective dose is 0.5 mg/kg to 1.5 mg/kg.

10. A method for preventing or treating a disease in an animal, wherein the method comprises administering an effective dose of a compound or a pharmaceutically acceptable salt, hydrate, solvate, racemate, enantiomer, diastereomer, or polymorph thereof to the animal; the compound has the structure of formula I: 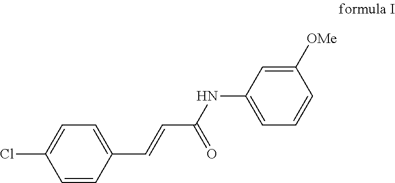 [SMILES: COc1cccc(NC(=O)/C=C/c2ccc(Cl)cc2)c1]
the disease includes at least one of a neurodegenerative disease, an intestinal permeability disease, and an inflammatory disease.

11. The method according to claim 10, wherein the neurodegenerative diseases include at least one of Alzheimer's disease, Parkinson's disease, multiple sclerosis, amyotrophic lateral sclerosis, spinocerebellar ataxia, and dementia of Pick's disease.

12. The method according to claim 10, wherein the intestinal permeability diseases include at least one of inflammatory bowel disease, irritable bowel syndrome, celiac disease, obesity, non-alcoholic fatty liver disease, type 2 diabetes, cardiovascular disease, and cancer.

13. The method according to claim 10, wherein the inflammatory diseases include at least one of neuroinflammation, inflammatory arthropathy, allergy, autism, anxiety, depression, and chronic fatigue syndrome; optionally, the neuroinflammation includes brain neuroinflammation.

14. The method according to claim 10, wherein the animal includes a mammal.

15. The method according to claim 10, wherein the animal includes a primate.

16. The method according to claim 10, wherein the animal includes a human being.

17. The method according to claim 10, wherein a mode of administration includes at least one of oral administration and injection administration.

18. The method according to claim 10, wherein the pharmaceutically acceptable excipient includes at least one of a carrier, a diluent, a stabilizer, a colorant, a solvent, a chelating agent, a dispersant, a preservative, an antifreeze agent, a thickener, a pH regulator, a protective agent, and a tension regulator.

19. The method according to claim 10, wherein the compound or the pharmaceutically acceptable salt, hydrate, solvate, racemate, enantiomer, diastereomer, or polymorph thereof is administered as a pharmaceutical composition; the pharmaceutical composition further comprises a pharmaceutically acceptable excipient.

20. The method according to claim 19, wherein the pharmaceutical composition is any one of a liquid preparation, a solid preparation, a sustained-release preparation, and a controlled-release preparation.
